(19) United States
(12) Patent Application Publication
(10) Pub. No.: US 2004/0109832 A1
(43) Pub. Date: Jun. 10, 2004

(54) Di-substituted resorcinols as skin lightening agents

(75) Inventors: Bijan Harichian, Warren, NJ (US); Michael James Barratt, Oak Ridge, NJ (US); Carol Annette Bosko, Oradell, NJ (US)

Correspondence Address: UNILEVER, PATENT DEPARTMENT, 45 RIVER ROAD, EDGEWATER, NJ, 07020, US

(73) Assignee: Unilever Home & Personal Care USA, Division Of Conopco, Inc.

(21) Appl. No.: 10/314,627

(22) Filed: Dec. 9, 2002

(51) Int. Cl. A61K 7/42; A61K 7/135; A61K 7/021

(52) U.S. Cl. 424/62; 424/59

(57) ABSTRACT

Cosmetic compositions and methods of skin lightening using compounds of formula I as skin lightening agents:

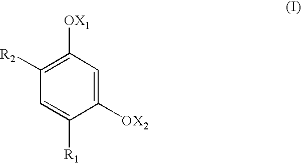

Where:

each X₁ and/or X₂, independently, is ═H or COR, CO₂R, CONHR groups esented by the following formula A:

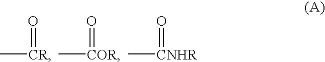

where R=C₁-C₁₈ saturated or unsaturated linear or branched hydrocarbon, and

each R₁ and/or R₂, independently is a C₁-C₁₈ saturated or unsaturated, linear or branched, hydrocarbon group.

### FIELD OF THE INVENTION

The invention relates to cosmetic methods of using resorcinol derivative compounds and cosmetic compositions including same, and more specifically, 4,6-di-substituted resorcinol derivatives, as skin lightening agents.

### BACKGROUND OF THE INVENTION

Many people are concerned with the degree of pigmentation of their skin. For example, people with age spots or freckles may wish such pigmented spots to be less pronounced. Others may wish to reduce the skin darkening caused by exposure to sunlight or to lighten their natural skin color. To meet this need, many attempts have been made to develop products that reduce the pigment production in the melanocytes. However, the substances identified thus far tend to have either low efficacy or undesirable side effects, such as, for example, toxicity or skin irritation. Therefore, there is a continuing need for new skin lightening agents, with improved overall effectiveness.

Resorcinol derivatives have cosmetic skin and hair benefits. Certain resorcinol derivatives, particularly 4-substituted resorcinol derivatives, are useful in cosmetic compositions for skin lightening benefits. Resorcinol derivatives are described in many publications, including Hu et al., U.S. Pat. No. 6,132,740; Collington et al., PCT Patent Application WO 00/56702; Bradley et al., European Patent Application EP 1 134 207; Shinomiya et al., U.S. Pat. No. 5,880,314; LaGrange et al., U.S. Pat. No. 5,468,472; Hiroaki et al., Japanese Patent Application JP11-255638 A2; Torihara et al., U.S. Pat. No. 4,959,393; and Japanese published patent applications JP 2001-010925 and JP2000-327557. Resorcinol derivatives are known compounds and can be readily obtained by various means, including by a method wherein a saturated carboxylic acid and resorcinol are condensed in the presence of zinc chloride and the resultant condensate is reduced with zinc amalgam/hydrochloric acid (Lille, et al., Tr. Nauch-Issled. Inst. Slantsev 1969, No. 18:127-134), or by a method wherein resorcinol and a corresponding alkyl alcohol are reacted in the presence of an alumina catalyst at a high temperature of from 200 to 400° C. (British Patent No. 1,581,428). Some of these compounds can be irritating to the skin.

Applicants have now discovered that the use of 4,6-di-substituted resorcinol derivative compounds and compositions including the compounds delivers skin lightening benefits. The general chemical formulas and structures of these compounds are discussed in more detail herein below. The 4,6-disubstituted resorcinol derivative compounds have been found to be effective and possibly less irritating to the skin and have not been previously used for lightening skin.

### SUMMARY OF THE INVENTION

The use of compounds of the general formula I, and compositions including same, delivers skin lightening benefits with potential reduced irritation. The present invention provides a cosmetic composition and method of skin lightening using in addition to a cosmetically acceptable vehicle, about 0.000001 to about 50% of a compound of formula I,

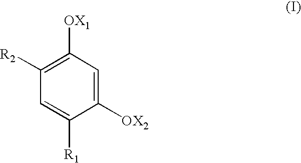

Where:

each X₁ and/or X₂, independently, is ═H or COR (acyl group), CO₂R , CONHR groups, the latter three represented by the following formula A, respectively:

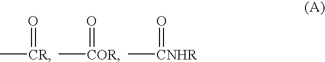

where R=C₁-C₁₈ saturated or unsaturated, linear, branched or cyclic hydrocarbon, and

each R₁ and/or R₂, independently is a C₁-C₁₈ saturated or unsaturated, linear, branched or cyclic, hydrocarbon group.

In a preferred embodiment, each or both X₁ and/or X₂ represents H. In a more preferred embodiment, both X₁ and X₂ represent H, so that the compound is of formula II as follows (with R₁ and R₂ defined as above with reference to formula I):

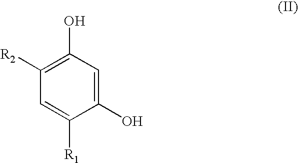

In a most preferred embodiment, in the formula II above, both R₁ and R₂ represent an isopropyl group.

Optionally, the hydroxy groups may be further substituted by methods known in the art. For example, the one or both hydroxy groups may be esterified with any or a combination of the following acids: ferulic acid, vanillic acid, sebacic acid, azaleic acid, benzoic acid, caffeic acid, coumaric acid, salicylic acid, cysteine, cystine, lactic acid, and glycolic acid.

Further skin benefit agents may be included in the compositions useful for the inventive method. Organic and inorganic sunscreens may also be included.

The inventive compositions and methods have effective skin lightening properties, may be less irritating to the skin, and are cost-effective.

### DETAILED DESCRIPTION OF THE INVENTION

As used herein, the term “cosmetic composition” is intended to describe compositions for topical application to human skin.

The term “skin” as used herein includes the skin on the face, neck, chest, back, arms, axilla, hands, legs, and scalp.

Except in the examples, or where otherwise explicitly indicated, all numbers in this description indicating amounts of material or conditions of reaction, physical properties of materials and/or use are to be understood as modified by the word “about”. All amounts are by weight of the composition, unless otherwise specified.

It should be noted that in specifying any range of concentration, any particular upper concentration can be associated with any particular lower concentration.

For the avoidance of doubt the word “comprising” is intended to mean including but not necessarily consisting of or composed of. In other words the listed steps or options need not be exhaustive.

### SKIN LIGHTENING AGENTS

4,6-Di-Substituted Resorcinol Derivatives

The invention is concerned with the use of compounds of general formula I, shown below, and compositions including same, as skin lightening agents. A particular advantage of the inventive compositions and methods is that compounds of general formula I can be less irritating to the skin than other, known, skin lightening compounds. Additionally, compounds of general formula I are relatively simple and cost-effective to manufacture. The present invention provides a cosmetic composition and method of skin lightening using in addition to a cosmetically acceptable vehicle, about 0.000001 to about 50% of a compound of formula I,

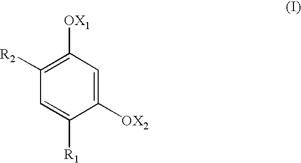

Where:

each X₁ and/or X₂, independently, is ═H or COR, CO₂R , CONHR groups, the latter three represented by the following formula A, respectively:

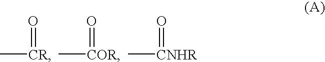

where R=C₁-C₁₈ saturated or unsaturated linear, branched or cyclic hydrocarbon, and

each R₁ and/or R₂, independently is a C₁-C₁₈ saturated or unsaturated, linear, branched or cyclic hydrocarbon group.

In a preferred embodiment, each or both X₁ and/or X₂ represents H. In a more preferred embodiment, both X₁ and X₂ represent H, so that the compound is of formula II as follows (with R₁ and R₂ defined as above with reference to formula I):

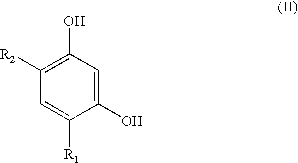

The most preferred embodiment may be prepared by reaction of resorcinol and isopropyl alcohol over an acidic catalyst, preferably sulfuric acid catalyst (starting materials are available from Yick-Vic Chemicals & Pharmaceuticals (HK) Ltd/Hong Kong), denoted by the following formula III:

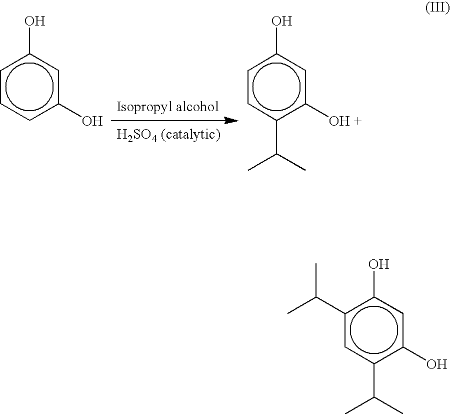

Similarly, for lower or higher chain R₁ and/or R₂ groups, the corresponding carbon chain length of alcohol would be used.

In the formula II, optionally, the hydroxy groups (the hydrogens on one or both of the OH-groups) may be further substituted by methods known in the art, such as esterification reaction of resorcinol with an acid anhydride. For example, the one or both hydroxy groups may be esterified with any or a combination of the following acids (or anhydrides thereof): ferulic acid, vanillic acid, sebacic acid, azaleic acid, benzoic acid, caffeic acid, coumaric acid, salicylic acid, cysteine, cystine, lactic acid, and glycolic acid.

Further skin benefit agents may be included in the compositions useful for the inventive method. For example, the composition may include a compound of general formula I in combination with a mono-substituted resorcinol derivative, such as 4-ethyl resorcinol, 4-isopropyl resorcinol, 4-butyl resorcinol, 4-hexyl resorcinol, and other resorcinol derivatives substituted at the 4-position. Organic and inorganic sunscreens, as well as fragrances, may also be included.

The inventive compositions and methods have effective skin lightening properties, may be less irritating to the skin than other skin lightening actives, and are relatively easy to manufacture and cost-effective.

The compositions generally contain about 0.000001 to about 50% of compounds of general formula I. Compounds of formula II are preferred, and compounds of formula II where both R₁ and R₂ represent an isopropyl group are most preferred. The amount of the compound of general formula I is preferably in the range of about 0.00001% to about 10%, more preferably about 0.001 to about 7%, most preferably from 0.01 to about 5%, of the total amount of a cosmetic composition.

### OPTIONAL SKIN BENEFIT AGENTS

Preferred cosmetic compositions are those suitable for the application to human skin according to the method of the present invention, which optionally include a skin benefit agent in addition to a compound of general formula I.

Suitable additional skin benefit agents include anti-aging, wrinkle-reducing, skin whitening, anti-acne, and sebum reduction agents. Examples of these include alpha-hydroxy acids, beta-hydroxy acids, polyhydroxy acids, hydroquinone, t-butyl hydroquinone, Vitamic C derivatives, dioic acids (e.g., malonic acid, sebacic acid), retinoids, niacinamide, linoleic acid, conjugated linoleic acid, and resorcinol derivatives other than compound of formula I of the present invention.

### COSMETICALLY ACCEPTABLE CARRIER

The cosmetically acceptable vehicle may act as a dilutant, dispersant or carrier for the skin benefit ingredients in the composition, so as to facilitate their distribution when the composition is applied to the skin.

The vehicle may be aqueous, anhydrous or an emulsion. Preferably, the compositions are aqueous or an emulsion, especially water-in-oil or oil-in-water emulsion, preferably oil in water emulsion. Water when present will be in amounts which may range from 5 to 99%, preferably from 20 to 70%, optimally between 40 and 70% by weight.

Besides water, relatively volatile solvents may also serve as carriers within compositions of the present invention. Most preferred are monohydric C₁-C₃ alkanols. These include ethyl alcohol, methyl alcohol and isopropyl alcohol. The amount of monohydric alkanol may range from 1 to 70%, preferably from 10 to 50%, optimally between 15 to 40% by weight.

Emollient materials may also serve as cosmetically acceptable carriers. These may be in the form of silicone oils and synthetic esters. Amounts of the emollients may range anywhere from 0.1 to 50%, preferably between 1 and 20% by weight.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile emollients useful in the present compositions are the polydimethyl siloxanes having viscosities from about 10 to about 400 centistokes at 25° C.

Among the ester emollients are:

(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.

(2) Ether-esters such as fatty acid esters of ethoxylated fatty alcohols.

(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.

(4) Wax esters such as beeswax, spermaceti, myristyl myristate, stearyl stearate and arachidyl behenate.

(5) Sterol esters, of which cholesterol fatty acid esters are examples.

Fatty acids having from 10 to 30 carbon atoms may also be included as cosmetically acceptable carriers for compositions of this invention. Illustrative of this category are pelargonic, lauric, myristic, palmitic, stearic, isostearic, hydroxystearic, oleic, linoleic, ricinoleic, arachidic, behenic and erucic acids.

Humectants of the polyhydric alcohol-type may also be employed as cosmetically acceptable carriers in compositions of this invention. The humectant aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant may range anywhere from 0.5 to 30%, preferably between 1 and 15% by weight of the composition.

Thickeners may also be utilized as part of the cosmetically acceptable carrier of compositions according to the present invention. Typical thickeners include crosslinked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0001 to 5%, usually from 0.001 to 1%, optimally from 0.01 to 0.5% by weight.

Collectively the water, solvents, silicones, esters, fatty acids, humectants and/or thickeners will constitute the cosmetically acceptable carrier in amounts from 1 to 99.9%, preferably from 80 to 99% by weight.

An oil or oily material may be present, together with an emulsifier to provide either a water-in-oil emulsion or an oil-in-water emulsion, depending largely on the average hydrophilic-lipophilic balance (HLB) of the emulsifier employed.

Surfactants may also be present in cosmetic compositions of the present invention. Total concentration of the surfactant will range from 0.1 to 40%, preferably from 1 to 20%, optimally from 1 to 5% by weight of the composition. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C₈-C₂₀ acyl isethionates, acyl glutamates, C₈-C₂₀ alkyl ether phosphates and combinations thereof.

### OPTIONAL COMPONENTS

In the cosmetic compositions of the invention, there may be added various medically effective ingredients, such as allantoin, a placenta extract; other thickeners, plasticizers; calamine; pigments; antioxidants; and chelating agents; as well as sunscreens, including organic and inorganic sunscreens. Typical organic sunscreens are PARSOL 1789 and PARSOL MCX.

Other adjunct minor components may also be incorporated into the cosmetic compositions. These ingredients may include coloring agents, opacifiers, and perfumes. Amounts of these other adjunct minor components may range anywhere from 0.001% up to 20% by weight of the composition.

Sunscreens

For use as sunscreen, metal oxides may be used alone or in mixture and/or in combination with organic sunscreens. Examples of organic sunscreens include but are not limited those set forth in the table below:

TABLE 1
Typical Organic Sunscreens

| CTFA Name | Trade Name | Supplier |
|---|---|---|
| Benzophenone-3 | UVINUL M-40 | BASF Chemical Co. |
| Benzophenone-4 | UVINUL MS-40 | BASF Chemical Co. |
| Benzophenone-8 | SPECRA-SORB UV-24 | American Cyanamide |
| DEA | | |
| Methoxycinnamate | BERNEL HYDRO | Bernel Chemical |
| Ethyl dihydroxypropyl-PABA | AMERSCREEN P | Amerchol Corp. |
| Glyceryl PABA | NIPA G.M.P.A. | Nipa Labs. |
| Homosalate | KEMESTER HMS | Hunko Chemical |
| Methyl anthranilate | SUNAROME UVA | Felton Worldwide |
| Octocrylene | UVINUL N-539 | BASF Chemical Co. |
| Octyl dimethyl PABA | AMERSCOL | Amerchol Corp. |
| Octyl methoxycinnamate | PARSOL MCX | Bernel Chemical |
| Octyl salicylate | SUNAROME WMO | Felton Worldwide |
| PABA | PABA | National Starch |
| 2-Phenylbenzimidazole-5-sulphonic acid | EUSOLEX 232 | EM Industries |
| TEA salicylate | SUNAROME W | Felton Worldwide |
| 3-(4-methylbenzylidene)-camphor | EUSOLEX 6300 | EM Industries |
| Benzophenone-1 | UVINUL 400 | BASF Chemical Co. |
| Benzophenone-2 | UVINUL D-50 | BASF Chemical Co. |
| Benzophenone-6 | UVINUL D-49 | BASF Chemical Co. |
| Benzophenone-12 | UVINUL 408 | BASF Chemical Co. |
| 4-Isopropyl dibenzoyl methane | EUSOLEX 8020 | EM Industries |
| Butyl methoxy dibenzoyl methane | PARSOL 1789 | Givaudan Corp. |
| Etocrylene | UVINUL N-35 | BASF Chemical Co. |

The amount of the organic sunscreens in the cosmetic composition is preferably in the range of about 0.1 wt % to about 10 wt %, more preferably about 1 wt % to 5 wt %.

Preferred organic sunscreens are PARSOL MCX and Parsol 1789, due to their effectiveness and commercial availability.

Perfumes

Perfumes are fragrance compositions that are mixtures of components providing, usually, a pleasing sense of smell. Terpenes and terpene derivatives are often an important component of fragrances. Fragrance terpenes and derivatives are described in Bauer, K., et al., Common Fragrance and Flavor Materials, VCH Publishers (1990).

Terpenes and derivatives that may preferably be incorporated in the inventive cosmetic compositions are divided into three classes, including acyclic terpenoids, cyclic terpenoids, and cycloaliphatic compounds that are structurally related to terpenoids.

Terpene derivatives within each of the three classes include alcohols, ethers, aldehydes, acetals, acids, ketones, esters, and terpene compounds that contain heteroatoms such as nitrogen or sulfur.

Examples of terpenes and derivative that may be incorporated in the cosmetic compositions of the present invention are set forth in the tables below:

TABLE 2
Acyclic Terpenes and Derivatives

| | |
|---|---|
| HYDROCARBONS | |
| Myrcene | |
| Ocimene | |
| beta-Farnesene | |
| ALCOHOLS | |
| Dihydromyrcenol | (2,6-dimethyl-7-octen-2-ol) |
| Geraniol | (3,7-dimethyl-trans-2,6-octadien-1-ol) |
| Nerol | (3,7-dimethyl-cis-2,6-octadien-1-ol) |
| Linalool | (3,7-dimethyl-1,6-octadien-3-ol) |
| Myrcenol | (2-methyl-6-methylene-7-octen-2-ol) |
| Lavandulol | |
| Citronellol | (3,7-dimethyl-6-octen-1-ol) |
| Trans-trans-Farnesol | (3,7,11-trimethyl-2,6,10-dodecatrien-1-ol) |
| Trans-Nerolidol | (3,7,11-trimethyl-1,6,10-dodecatrien-3-ol) |
| ALDEHYDES | |
| AND ACETALS | |
| Citral | (3,7-dimethyl-2,6-octadien-1-al) |
| Citral diethyl acetal | (3,7-dimethyl-2,6-octadien-1- |
| | al diethyl acetal) |
| Citronellal | (3,7-dimethyl-6-octen-1-al) |
| Citronellyloxyacetaldehyde | |
| 2,6,10-Trimethyl-9-undecenal | |
| KETONES | |
| Tagetone | |
| Solanone | |
| Geranylacetone | (6,10-dimethyl-5,9-undecadien-2-one) |

ACIDS AND ESTERS
Cis-Geranic acid
Citronellic acid
Geranyl Esters, including Geranyl formate,
Geranyl acetate, Geranyl propionate,
Geranyl isobutyrate, Geranyl isovalerate
Neryl Esters, including Neryl acetate
Linalyl Esters, including Lynalyl formate,
Linalyl acetate, Linalyl propionate,
Linalyl butyrate, Linalyl isobutyrate,
Lavandulyl Esters, including Lavendulyl acetate
Citronellyl Esters, including Citronellyl formate,
Citronellyl acetate, Citronellyl
propionate, Citronellyl isobutyrate,
Citronellyl isovalerate, Citronellyl tiglate
NITROGEN CONTAINING UNSATURATED
TERPENE DERIVATIVES
Cis-Geranic acid nitrile
Citronellic acid nitrile

TABLE 3
Cyclic Terpenes and Derivatives

| | |
|---|---|
| HYDROCARBONS | |
| Limonene | (1,8-p-menthadiene) |
| Alpha-Terpinene | |
| Gamma-Terpinene | (1,4-p-menthadiene) |
| Terpinolene | |
| Alpha-Phellandrene | (1,5-p-menthadiene) |
| Beta-Phellandrene | |
| Alpha-Pinene | (2-pinene) |
| Beta-Pinene | (2(10)-pinene) |
| Camphene | |
| 3-Carene | |
| Caryophyllene | |
| (+)-Valencene | |
| Thujopsene | |
| Alpha-Cedrene | |
| Beta-Cedrene | |
| Longifolene | |
| ALCOHOLS | |
| AND ETHERS | |
| (+)-Neoiso-isopulegol | |
| Isopulegol | (8-p-menten-3-ol) |
| Alpha-Terpineol | (1-p-menten-8-ol) |
| Beta-Terpineol | |
| Gamma-Terpineol | |
| Delta-Terpineol | |
| 1-Terpinen-4-ol | (1-p-menten-4-ol) |

Cyclic Terpenoids

| | |
|---|---|
| ALDEHYDES | |
| AND KETONES | |
| Carvone | (1,8-p-mantadien-6-one) |
| Alpha-lonone | (C₁₃H₂₀O) |
| Beta-lonone | (C₁₃H₂₀O) |
| Gamma-lonone | (C₁₃H₂₀O) |
| Irone, alpha-, | (C₁₄H₂₂O) |
| beta-, gamma- | |
| n-Methylionone, | (C₁₄H₂₂O) |
| alpha-, beta-, gamma- | |
| Isomethylionone, | (C₁₄H₂₂O) |
| alpha-, beta-, gamma- | |
| Allylionone | (C₁₆H₂₄O) |
| Pseudoionone | |
| n-Methylpseudoionone | |
| Isomethylpseudoionone | |
| Damascones | 1-(2,6,6-trimethylcyclohexenyl)- |
| | 2-buten-1-ones |
| Including beta-Damascenone | 1-(2,6,6-trimethyl- |
| | 1,3-cyclohadienyl)- |
| | 2-buten-1-one |
| Nootkatone | 5,6-dimethyl-8- |
| | isopropenylbicyclo[4.4.0]-1- |
| | decen-3-one |
| Cedryl methyl ketone | (C₁₇H₂₆O) |
| ESTERS | |
| Alpha-Terpinyl acetate | (1-p-menthen-8-yl acetate) |
| Nopyl acetate | (−)-2-(6,6-dimethylbicyclo[3.1.1]hept- |
| | 2-en-2-yl)ethyl acetate |
| Khusymil acetate | |

TABLE 4
Cycloaliphatic Compounds Structurally Related to Terpenes

ALCOHOLS
5-(2,2,3-Trimethyl-3-cyclopenten-1-yl)-3-methylpentan-2-ol
ALDEHYDES
2,4-Dimethyl-3-cyclohexene carboxaldehyde
4-(4-Methyl-3-penten-1-yl)-3-cyclohexene carboxaldehyde
4-(4-Hydroxy-4-methypentyl)-3-cyclohexene carboxaldehyde
KETONES
Civetone

| | |
|---|---|
| Dihydrojasmone | (3-methyl-2-pentyl-2-cyclopenten-1-one) |
| Cis-Jasmone | 3-methyl-2-(2-cis-penten-1-yl)-2-cyclopenten-1- |
| | one |

5-Cyclohexadecen-1-one
2,3,8,8-Tetramethyl-1,2,3,4,5,6,7,8-octahydro-2-napthalenyl methyl
ketone 3-methyl-2-cyclopenten-2-ol-1-one
ESTERS
4,7-Methano-3a,4,5,6,7,7a-hexahydro-5-(or 6)-indenyl acetate
Allyl 3-cyclohexylpropionate
Methyl dihydrojasmonate methyl (3-oxo-pentylcyclopentyl)acetate

Preferably, the amount of terpenes and derivatives in the cosmetic composition is in the range of about 0.000001% to about 10%, more preferably about 0.00001% to about 5 wt %, most preferably about 0.0001% to about 2%.

### USE OF THE COMPOSITION

The method according to the invention is intended primarily as using a personal care product for topical application to human skin.

In use, a small quantity of the composition, for example from 1 to 5 ml, is applied to exposed areas of the skin, from a suitable container or applicator and, if necessary, it is then spread over and/or rubbed into the skin using the hand or fingers or a suitable device.

### PRODUCT FORM AND PACKAGING

The cosmetic composition useful for the method of the invention can be formulated as a lotion having a viscosity of from 4,000 to 10,000 mPas, a fluid cream having a viscosity of from 10,000 to 20, 000 mPas or a cream having a viscosity of from 20,000 to 100,000 mPas, or above. The composition can be packaged in a suitable container to suit its viscosity and intended use by the consumer. For example, a lotion or fluid cream can be packaged in a bottle or a roll-ball applicator or a propellant-driven aerosol device or a container fitted with a pump suitable for finger operation. When the composition is a cream, it can simply be stored in a non-deformable bottle or squeeze container, such as a tube or a lidded jar. When the composition is a solid or semi-solid stick, it may be packaged in a suitable container for manually or mechanically pushing out or extruding the composition.

The invention accordingly also provides a closed container containing a cosmetically acceptable composition as herein defined.

The following examples are by way of example, not by way of limitation, of the principles of the present invention, to illustrate the best mode of carrying out the invention.

### EXAMPLE 1

The following compounds, which were prepared by the method described above (formula III) and purified by HPLC, were used throughout the examples that follow:

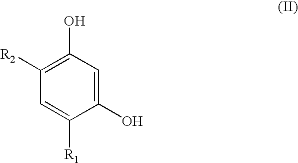

The inventive compound where both R₁ and R₂ are isopropyl groups, is referred to as 4,6-di-isopropyl resorcinol. The compound where R₁ represents an isopropyl group and R₂ represent H is referred to as 4-isopropyl resorcinol.

The 4-isopropyl resorcinol and the 4,6-di-isopropyl resorcinol were purified to greater than 98% purity (as confirmed by gas chromatography) by the following HPLC method.

HPLC System was composed of:

Waters 600 Pump Controller

717+ Autosampler

Waters 996 Diode Array UV/VIS Detector

Waters Fraction Collector

HPLC Column: from Phenomenex

A 250×21.2 mm Sphereclone ODS(2) Reversed Phase Column, 5 micron particle size used as packing.

A linear gradient, over 30 min, of 70/30 water/acetonitrile to 50/50 water/acetonitrile.

The flow rate used to conduct the separation was constant at 10 mL/min. The detector wavelength used was 280 nm.

The elution times were: 15 minutes for the 4-isopropyl resorcinol and 29 minutes for the 4,6-di-isopropyl resorcinol.

At the time a given molecule was found to elute, the fraction collector directed the eluent into separate catch flasks so that each compound could be individually saved.

Purity of the resorcinol derivatives was assessed by gas chromatography. Samples (10 mg) were derivatized with 500 ul of pyridine and 300 ul of bis(trimethyl silyl)trifluoroacetate amide (Regis Chemical, Morton Grove, II) at 70 oC for 30 min. 1 uL was injected onto a Hewlett-Packard gas chromatograph with an HP1 crosslinked methyl silicone column (25 m×0.2 mm). Derivatized resorcinols were volatilized from the column through a temperature gradient of 60 oC to 180 oC for 20 min.

### EXAMPLE 2

Cosmetic compositions within the scope of the invention were prepared.

A base formulation, shown in the Table below, was made by heating phase A ingredients to 70 to 85° C. with stirring. Phase B ingredients were heated in a separate container to 70 to 85° C. with stirring. Then, phase A was added into phase B while both phases were kept at 70 to 85° C. The mixture was stirred for at least 15 minutes at 70 to 85° C., then cooled.

TABLE 5

| | a | b | |
|---|---|---|---|
| Ingredients | % wt. | % wt. | Phase |
| Isostearyl Palmitate | 6.00 | 6.00 | A |
| C12-C15 Alkyl Octanoate | 3.00 | 3.00 | A |
| PEG-100 Stearate | 2.00 | 2.00 | A |
| Glyceryl Hydroxystearate | 1.50 | 1.50 | A |
| Stearyl Alcohol | 1.50 | 1.50 | A |
| Stearic acid | 3.00 | 4.00 | A |
| TEA, 99% | 1.20 | 1.20 | B |
| Dimethicone | 1.00 | 1.00 | A |
| Sorbitan Monostearate | 1.00 | 1.00 | A |
| Magnesium Aluminum Silicate | 0.60 | 0.60 | B |
| Vitamin E acetate | 0.10 | 0.10 | A |
| Cholesterol | 0.50 | 0.50 | A |
| Simethicone | 0.01 | 0.01 | B |
| Xanthan gum | 0.20 | 0.20 | B |
| Hydroxyethylcellulose | 0.50 | 0.50 | B |
| Propylparaben | 0.10 | 0.10 | B |
| Disodium EDTA | 0.05 | 0.05 | B |
| Butylated hydroxytolene | 0.05 | 0.05 | B |
| 4,6-di-isopropyl resorcinol | 0.05 | 2.00 | B |
| Niacinamide | 1.00 | 1.00 | B |
| Metal oxide | 2.50 | 5.00 | B |
| Methylparaben | 0.15 | 0.15 | B |
| Water | BAL* | BAL* | B |
| Total | 100.00 | 100.00 | B |

*BAL means Balance.

### EXAMPLE 3

Additional cosmetic compositions within the scope of the invention were prepared.

TABLE 6

| | Wt % | Phase |
|---|---|---|
| water, DI | BALANCE | A |
| disodium EDTA | 0.05 | A |
| magnesium aluminum silicate | 0.6 | A |
| methyl paraben | 0.15 | A |
| simethicone | 0.01 | A |
| butylene glycol 1,3 | 3.0 | A |
| hydroxyethylcellulose | 0.5 | A |
| glycerine, USP | 2.0 | A |
| xanthan gum | 0.2 | A |
| triethanolamine | 1.2 | B |
| stearic acid | 3.0 | B |
| propyl paraben NF | 0.1 | B |
| glyceryl hydroxystearate | 1.5 | B |
| stearyl alcohol | 1.5 | B |
| isostearyl palmitate | 6.0 | B |
| C12-15 alcohols octanoate | 3.0 | B |
| dimethicone | 1.0 | B |
| cholesterol NF | 0.5 | B |
| sorbitan stearate | 1.0 | B |
| Micronized titanium dioxide | 5.0 | C |
| tocopheryl acetate | 0.1 | B |
| PEG-100 stearate | 2.0 | B |
| sodium stearoyl lactylate | 0.5 | B |
| hydroxycaprylic acid | 0.1 | C |
| 4,6-di-isopropyl resorcinol | 10.0 | C |
| PARSOL MCX | 2.4 | C |
| alpha-bisabolol | 0.2 | C |

The composition of Example 3, was prepared as follows:

1. Heat Phase A to 80° C.

2. Heat Phase B to 75° C. in a separate container

3. Add B to A and mix with heat off for 30 min.

4. At 50° C. add Phase C and mix for 10 min.

### EXAMPLES 4-11

A set of additional compositions useful in the methods of the present invention were prepared within the scope of the present invention and are listed in the table below.

TABLE 7

| | | Examples (wt. %) | | | | | | | |
|---|---|---|---|---|---|---|---|---|---|
| | | 4 | | | | | | | |
| | | acid | | | | | | | |
| | | soap | | | | | | | |
| Ingredients | Phase | base | 5 | 6 | 7 | 8 | 9 | 10 | 11 |
| Stearic acid | A | 17.9 | 17.9 | 17.9 | 17.9 | 17.9 | 17.9 | 17.9 | 17.9 |
| Sodium cetearyl | A | | 2.2 | | 1 | 1.5 | 2 | 3 | 2 |
| sulfate* (emulsifier) | | | | | | | | | |
| Myrj 59* | A | | | 2 | 2 | 2 | 2 | 2 | 1 |
| (emulsifier) | | | | | | | | | |
| Span 60* | A | | | 2 | 2 | 2 | 2 | 2 | 1 |
| (emulsifiers) | | | | | | | | | |
| 4,6-di-isopropyl | B | 0.05 | 0.05 | 2.0 | 2.0 | 3.5 | 3.5 | 5.0 | 10.0 |
| resorcinol | | | | | | | | | |
| Micronized Zinc | B | 2.50 | 5.00 | 5.00 | 2.50 | 2.50 | 5.00 | 2.50 | 5.00 |
| Oxide | | | | | | | | | |
| KOH, 22% (form in situ | | 2.20 | | | | | | | |
| soap with | | | | | | | | | |
| stearic acid) | | | | | | | | | |
| Octyl | | 2.50 | | | 2.50 | 2.50 | | 2.50 | |
| methoxycinnamate | | | | | | | | | |
| Water | B | | BAL | BAL | BAL | BAL | BAL | BAL | BAL |
| Glycerin | B | 1 | 1 | 1 | 1 | 1 | 1 | 1 | 1 |

### EXAMPLE 12

### Mushroom Tyrosinase Assay

Mushroom tyrosinase inhibition is indicative of reduction in melanin synthesis, thereby showing skin lightening effect. This experiment shows the skin lightening efficacy of the resorcinol derivatives of the present invention.

Into each well of a 96-well plate, 150 microliters of phosphate buffer (100 mM, pH 7.0), 10 microliters of L-DOPA (L-3,4-Dihydroxyphenylalanine, 10 mM), and 20 microliters of skin lightening agent (dissolved in ethanol, which is the control) were added. Following an initial measurement of background absorbency at 475-nm, 20 microliters of mushroom tyrosinase (Sigma T-7755; 6050 units/ml) was added and incubated at room temperature.

Absorbency is read at 475-nm over the following time points: 0, 2, 4, and 6.5 minutes. The data is plotted as 475-nm absorbency vs. time (minutes) and the slope of the line is calculated (ΔAbs 475 nm/ min). Values are expressed as the percentage of the respective untreated ethanol control for melanin synthesis reaction.

$$\% of Control = \frac{( Reaction rate for treated reaction )}{( Reaction rate for untreated control )} \times 100 \%$$

The following Table shows the tyrosinase assay results for the skin lightening compounds 4-isopropyl resorcinols and 4,6-di-isopropyr resorcinol at a range of condentrations. The IC50 value refers to the skin lightener concentration that results in 50% tyrosinase inhibition relative to the control (with a goal being obtaining maximum activity at minimum concentration).

TABLE 8

| | |
|---|---|
| Concentration (micro M) | Percent of Control |

A. 4-Isopropyl Resorcinol IC50 ˜ 50 nM

| | |
|---|---|
| 100 | 30.4 |
| 50 | 28.7 |
| 10 | 28.7 |
| 1 | 26.9 |
| 0.5 | 28.7 |
| 0.25 | 30.4 |
| 0.125 | 35.8 |
| 0.0625 | 46.6 |
| 0.0312 | 60.9 |
| 0.0156 | 80.6 |

B. 4,6-Diisopropyl Resorcinol IC50 = 750 nM

| | |
|---|---|
| 100 | 39.4 |
| 50 | 34.0 |
| 10 | 28.7 |
| 1 | 48.4 |
| 0.5 | 60.9 |
| 0.25 | 75.2 |
| 0.125 | 86.0 |
| 0.0625 | 93.1 |
| 0.0312 | 94.9 |
| 0.0156 | 96.7 |

C. 4-Ethyl Resorcinol IC50 = 350 nM

| | |
|---|---|
| 100 | 18 |
| 50 | 21 |
| 10 | 25 |
| 1 | 32 |
| 0.5 | 43 |
| 0.25 | 57 |
| 0.125 | 64 |
| 0.0625 | 72 |
| 0.0312 | 82 |
| 0.0156 | 86 |

The following Table shows comparative results for inhibition of tyrosinase (which in turn inhibits melanin synthesis) for 4-isopropyl resorcinol, 4-ethyl resorcinol, 4,6-di-isopropyl resorcinol, 4,5-dimethyl resorcinol and resorcinol.

TABLE 9

| | Concentration | % of Control |
|---|---|---|
| Compound | (micro M) | (Melanin Synthesis) |
| 4-Isopropyl Resorcinol | 1 | 27 |
| | 10 | 29 |
| | 50 | 29 |
| | 100 | 30 |
| 4-Ethyl Resorcinol | 1 | 32 |
| | 10 | 25 |
| | 50 | 21 |
| | 100 | 18 |
| 4,6-di-Isopropyl | 1 | 48 |
| Resorcinol | 10 | 29 |
| | 50 | 34 |
| | 100 | 39 |
| 4,5-di-Methyl Resorcinol | 1 | 95 |
| | 10 | 51 |
| | 50 | 35 |
| | 100 | 15 |
| Resorcinol | 1 | 98 |
| | 10 | 98 |
| | 50 | 100 |
| | 100 | 100 |

The data show that the inventive compounds, 4,6-di-Isopropyl resorcinols are effective skin lightening compounds, as well as the as 4-Isopropyl resorcinol. 4,6-di-isopropyl resorcinol is comparable to 4-Ethyl Resorcinol. The 4,-5-di-Methyl Resorcinol is not as effective. The 4,6-di-isopropyl resorcinol is more effective than Resorcinol, which has no activity.

It should be understood that the specific forms of the invention herein illustrated and described are intended to be representative only. Changes, including but not limited to those suggested in this specification, may be made in the illustrated embodiments without departing from the clear teachings of the disclosure. Accordingly, reference should be made to the following appended claims in determining the full scope of the invention.

What is claimed is:

1. A cosmetic method of skin lightening comprising applying to the skin a composition comprising:
a. about 0.000001 to about 50% of a compound of general formula I 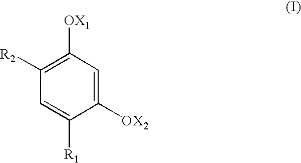
wherein each X₁ and/or X₂ independently, is selected from the group consisting of H, COR, CO₂R, and CONHR group; wherein R represents C₁-C₁₈ hydrocarbon;
each R₁ and/or R₂, independently is a C₁-C₁₈ hydrocarbon group; and
b. a cosmetically acceptable carrier.

2. The method of claim 1, wherein said composition further comprises a sunscreen.

3. The method of claim 2, wherein said sunscreen is a micronized metal oxide.

4. The method of claim 1, wherein said compound is a compound of formula II:

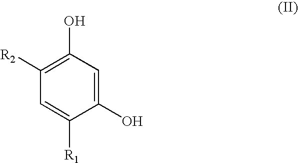

5. The method of claim 1, wherein said composition further comprises a fragrance.

6. The cosmetic method according to claim 1, wherein said composition further comprises a skin benefit agent selected from the group consisting of alpha-hydroxy acids, beta-hydroxy acids, polyhydroxy acids, hydroquinone, t-butyl hydroquinone, Vitamin C derivatives, dioic acids, retinoids, 4-substituted resorcinol derivatives, and mixtures thereof.

7. The cosmetic method of claim 1, wherein said composition further comprises an organic sunscreen selected from the group consisting of Benzophenone-3, Benzophenone-4, Benzophenone-8, DEA, Methoxycinnamate, Ethyl dihydroxypropyl-PABA, Glyceryl PABA, Homosalate, Methyl anthranilate, Octocrylene, Octyl dimethyl PABA, Octyl methoxycinnamate (PARSOL MCX), Octyl salicylate, PABA, 2-Phenylbenzimidazole-5-sulphonic acid, TEA salicylate, 3-(4-methylbenzylidene)-camphor, Benzophenone-1, Benzophenone-2, Benzophenone-6, Benzophenone-12, 4-Isopropyl dibenzoyl methane, Butyl methoxy dibenzoyl methane (PARSOL 1789), Etocrylene, and mixtures thereof.

8. A cosmetic composition comprising:
a. about 0.000001 to about 50% of a compound of general formula I 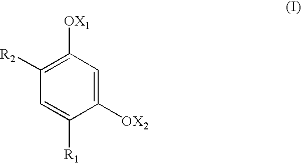
wherein each X₁ and/or X₂, independently, is selected from the group consisting of H, COR, CO₂R, and CONHR group; wherein R represents a C₁-C₁₈ hydrocarbon group;
each R₁ and/or R₂, independently is a C₁-C₁₈ saturated or unsaturated, linear or branched, hydrocarbon group; and
b. a cosmetically acceptable carrier.

9. The cosmetic composition of claim 8, wherein said compound is a compound of general formula II:

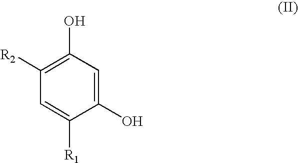

10. The cosmetic composition of claim 8, wherein said compound comprises about 0.00001% to about 10% of said composition.

11. The cosmetic composition of claim 8, wherein said compound comprises about 0.001% to about 7% of said composition.

12. The cosmetic composition of claim 8, wherein said compound comprises about 0.01% to about 5% of said composition.

13. The cosmetic composition of claim 8, further comprising a sunscreen.

14. The cosmetic composition of claim 8, wherein one or both of the hydroxyl groups is esterified with a carboxylic acid.

15. The cosmetic composition of claim 8, further comprising a 4-substituted resorcinol derivative.

16. The cosmetic composition of claim 15, wherein said 4-substituted resorcinol derivative is selected from the group consisting of 4-ethyl resorcinol, 4-isopropyl resorcinol, 4-butyl resorcinol. 4-hexyl resorcinol, and mixtures thereof.

17. The cosmetic composition of claim 8, wherein the hydroxy groups are esterified with an acid selected from the group consisting of ferulic acid, vanillic acid, sebacic acid, azaleic acid, benzoic acid, caffeic acid, coumaric acid, salicylic acid, cysteine, cystine, lactic acid, glycolic acid and mixtures thereof.
